(19) United States
(12) Patent Application Publication
(10) Pub. No.: US 2016/0101225 A1
(43) Pub. Date: Apr. 14, 2016

(54) DIALYSIS SYSTEM

(71) Applicant: Purewater Therapeutics, LLC, Longmont, CO (US)

(72) Inventors: Mark Forrest Smith, Longmont, CO (US); W. Mark Davis, Louisville, CO (US); Justin Charles Smith, Broomfield, CO (US)

(73) Assignee: Purewater Therapeutics, LLC

(21) Appl. No.: 14/860,387

(22) Filed: Sep. 21, 2015

Related U.S. Application Data
(60) Provisional application No. 62/063,300, filed on Oct. 13, 2014.

(51) Int. Cl. A61M 1/16; C02F 1/28

(52) U.S. Cl. CPC A61M 1/1696; A61M 1/1656; A61M 1/1686; C02F 1/283; C02F 2103/026

(57) ABSTRACT

A dialysis system is disclosed. An example dialysis system includes a heat disinfected dialysate fluid path. The example dialysis system also includes a disposable blood fluid path. The example dialysis system also includes an active carbon filter to purify influent water to purified water for dialysis or as a substitution fluid. An example method for dialysis includes providing a heat disinfected dialysate fluid path, providing a disposable blood fluid path, and purifying influent water via an active carbon filter to for dialysis or as a substitution fluid.

### CROSS REFERENCE TO RELATED APPLICATION

This application claims the priority benefit of U.S. Provisional Patent Application No. 62/063,300 filed Oct. 13, 2014 for “Hemodialysis Systems,” hereby incorporated by reference in its entirety as though fully set forth herein.

### BACKGROUND

Significant technical challenges exist in bringing dialysis treatment from the clinic to home use, including: water purification, cost of consumables, and differences in prescription between in-center, self-care, and at-home machines.

### BRIEF DESCRIPTION OF THE DRAWINGS

FIGS. 1-3 illustrate an example dialysis system as it may be implemented as an integrated dialysis machine (FIG. 1), an active carbon dialysate filter dialysis machine (FIG. 2), and a universal active carbon dialysate filter machine (FIG. 3).

FIG. 4 is an illustration of an example pre-treatment subsystem.

FIG. 5 is an illustration of an example Active Carbon Filter Subsystem.

FIG. 6 is an illustration of an example Dialysate Preparation subsystem.

FIG. 7 is an illustration of an example Ultrafiltrate Subsystem.

FIG. 8 is an illustration of an example Dialyzer Subsystem.

FIG. 9 is an illustration of an example Blood Handling Subsystem.

FIG. 10 is an illustration of an example Mechanical Subsystem.

FIG. 11 is an illustration of an example Electrical and Software Subsystem.

FIG. 12 shows an example active carbon dialysate filter subsystem of the active carbon dialysate filter dialysis machine.

FIG. 13 shows an example ultrafiltrate subsystem of the active carbon dialysate filter dialysis machine.

FIG. 14 shows an example active carbon dialysate filter subsystem of the Universal ACDF machine.

FIG. 15 shows an example ultrafiltrate subsystem of the Universal ACDF machine.

### DETAILED DESCRIPTION

Dialysis systems disclosed herein implement a novel water purification technology. The Dialysis systems can be used economically and easily in any of the in-center (i.e., clinic), self-care, acute, and at-home settings. The systems may implement either high flow single-pass hemodialysis, hemofiltration, continuous renal replacement therapy, intermittent hemodialysis, ultrafiltration, or hemodiafiltration. Nephrologists should not be concerned with changing prescriptions as therapy modality changes (e.g., self-care to at-home) because the machine is the same for both modalities. The machine may be heat-disinfected, and include an inexpensive blood-side disposable. The integrated water purification system accepts drinking water input and yields substitution fluid or water for dialysis. It can be manufactured to be small, light, and modular (e.g., enhancing depot repair). Remote monitoring of treatment may also be possible through an Ethernet interface. A portable system may also be provided, as it may include an on-demand, low energy, carry-on sized, light weight water purification system, e.g., for use at-home or in-center (clinic) or elsewhere dialysis.

Prior systems address the water purification problem with a slow batch purification system—annoying customers with an eight hour process that is prone to failure. In addition, the prior machine requires more frequent treatment due to low dialysate flow rates. Other products in development use reverse osmosis systems. The disadvantages of reverse osmosis are significant, including for example: high waste water, high power consumption, and high cost of ownership. Other systems use sorbent technology to purify water. Sorbent requires costly consumables and is challenging for nephrologists to prescribe. Other issues with the home hemodialysis machines in the market and in development include: high cost consumables, lack of mobility due to size and weight, and the inability to be used for all three common treatment modalities (in-center, self-care, and at-home).

Before continuing, it is noted that as used herein, the terms “includes” and “including” mean, but is not limited to, “includes” or “including” and “includes at least” or “including at least.” The term “based on” means “based on” and “based at least in part on.”

It is also noted that the examples described herein are provided for purposes of illustration, and are not intended to be limiting. Other devices and/or device configurations may be utilized to carry out the operations described herein.

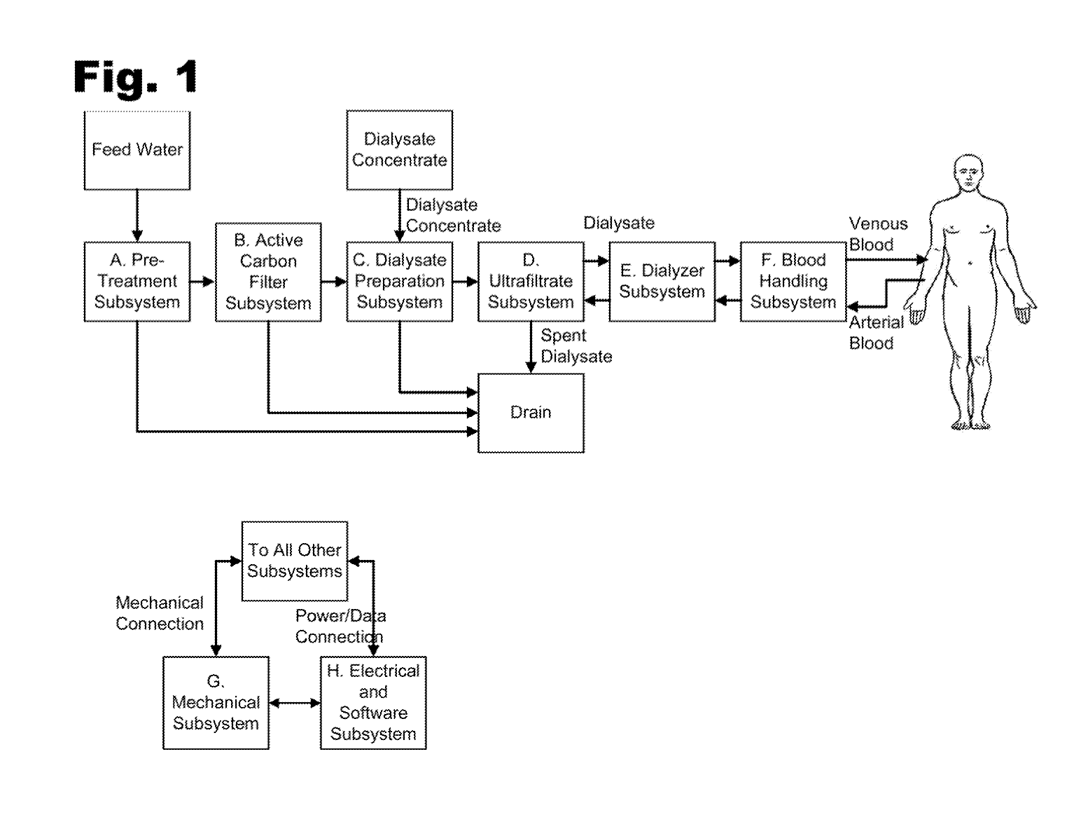

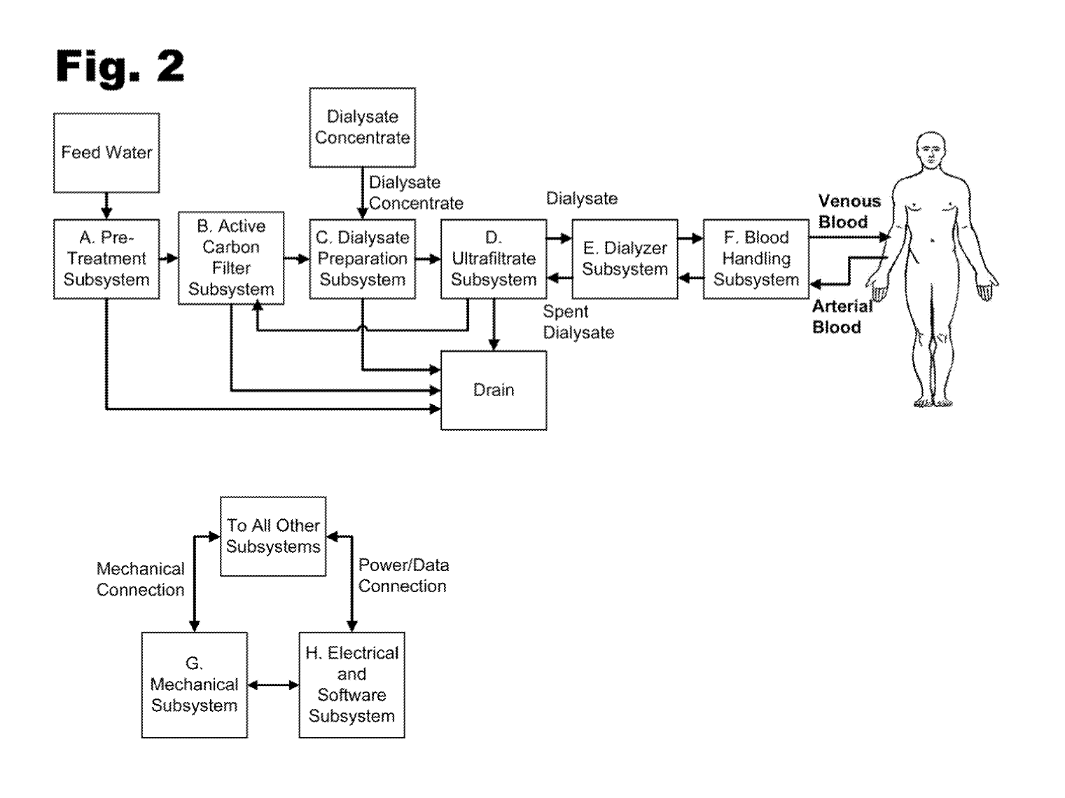

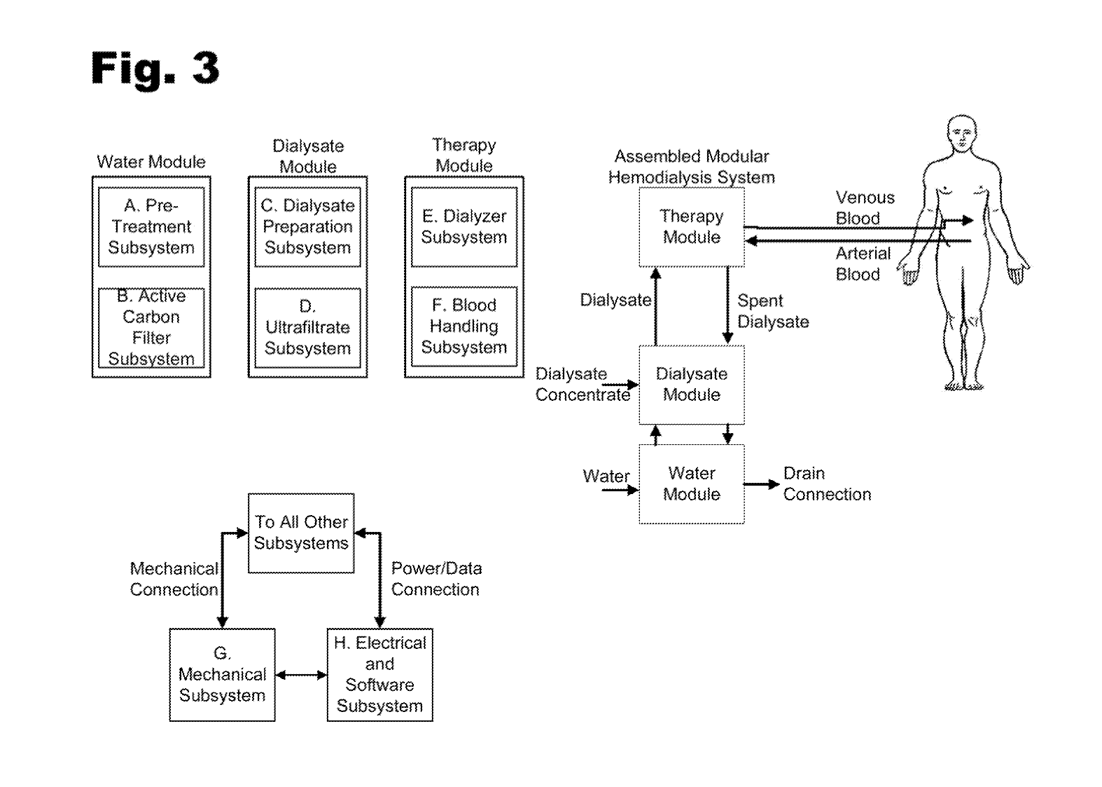

FIGS. 1-3 illustrate an example hemodialysis system as it may be implemented as an integrated hemodialysis machine (FIG. 1), an active carbon dialysate filter hemodialysis machine (FIG. 2), and a universal active carbon dialysate filter machine (FIG. 3).

The hemodialysis systems described herein provide a low waste, high efficiency water purification system for hemodialysis. In an example, the system provides a heat disinfected dialysate fluid path and a disposable blood fluid path. The systems are also capable of standard dialysis flow rates that are used in in-center dialysis machines, which means treatment only three times per week for the patients. The same machine may be used for in-center, self-care, acute, or at-home dialysis because it has all the features of an in-center machine, all the features required for at-home dialysis, an efficient water purification system, and an inexpensive disposable.

The hemodialysis systems described herein use an active carbon filter to purify water from drinking water to water for dialysis or substitution fluid. The hemodialysis systems described herein may be implemented as a single machine with water purification by active carbon filter and hemodialysis treatment. In an example, the system provides heat disinfection of a dialysis machine with the active carbon filter—the same component used for water purification.

In an example, the hemodialysis machine may be implemented as a modular hemodialysis machine. The modular hemodialysis system is an efficient way of producing hemodialysis machines for many environments, uses, and therapies in a capital efficient manner. With three types of modules (water, dialysate, and therapy) dialysis machines for acute, in-center, and at-home hemodialysis may be assembled based on custom needs.

The water module provides dialysis water to the dialysate module. In an example, the water module may be implemented for standard water purification, that is, it may be configured as system that provides water purification of drinking water to make heated dialysis water. The system removes chemical and biological contaminants. It handles normal drinking water and supplies the system with enough heated purified water to perform a standard hemodialysis treatment. This system may include subsystems: A. Pre-Treatment, and B. Active Carbon Filter.

In an example, the water module may be implemented for heavy water purification, that is, it may be configured as a system that provides water purification from heavily contaminated water to make heated dialysis water. The system removes up to one hundred times more biological and chemical contaminants than the standard water purification system. The system includes subsystems: A. pre-treatment, and B. active carbon filter.

In an example, the water module may be implemented for bagged dialysate, that is, it may be configured as a system that provides bagged dialysis fluid to the dialysate module with no further need for preparation. The system uses mass sensors for the bags to determine the flow rate of fluid from the bags. The fluid may be heated before it is passed to the dialysate module.

In an example, the water module may be implemented for purified water, that is, it may be configured as a system that provides pre-purified water to the Dialysate Module. This module may be useful in dialysis clinics that already have centralized water purification means. The module heats the purified water before passing it to the dialysate module.

The dialysate module mixes concentrated dialysate with purified water from the water module, provides volumetric control of dialysate to and from the dialyzer, and monitors ultrafiltrate removal from the patient.

In an example, the Dialysate Module may be implemented as a basic hemodialysis system. This system prepares dialysate, provides volumetric control of dialysate to and from the dialyzer, and manages ultrafiltration removal from the patient. Standard hemodialysis treatments are provided by this module. This system may include subsystems: C. Dialysate Preparation and D. Ultrafiltrate.

In an example, the Dialysate Module may be implemented as an advanced hemodialysis system. This system prepares dialysate, provides volumetric control of dialysate to and from the dialyzer, manages ultrafiltrate removal, and provides additional pumps, sensors, and controls for functionality needed to provide advanced treatments such as hemofiltration, hemodiafiltration, continuous renal replacement therapy, etc. This system may include subsystems: C. Dialysate Preparation and D. Ultrafiltrate.

The therapy module provides the treatment interface, including the blood-dialysis interface (dialyzer). The Therapy module may include the subsystems: E. Dialyzer. F. Blood-Handling, and elements of H. Electrical and Software. The Therapy Module's controller controls the Dialysate and Water Modules. The Mechanical Subsystem (G) may be split between the three modules. Mechanical and electrical connections between the modules provide pathways for fluids, data, and power.

The water, dialysate, and therapy modules may be combined in different ways for different applications, e.g., each providing functionality needed for a given set of circumstances. The following are example systems of how the modules listed above can be combined for hemodialysis machines. Others systems and modules may be created to address various needs.

In an example, the system may be implemented as a three part, modular hemodialysis system. The water modules allow for the use of raw water, purified water, or bagged dialysate on one machine. Dialysate modules can provide basic, as well as advanced blood therapy treatments (e.g., hemodialysis, hemodiafiltration, hemofiltration, continuous renal replacement therapy, intermittent hemodialysis, sustained low-efficiency dialysis, hemoperfusion, etc.). The system may include a common therapy module for all types of hemodialysis. A water purification module can provide on-line substitution quality fluid integrated with the hemodialysis machine.

In an example, a home hemodialysis machine with standard drinking water may include a water module for standard water purification, a dialysate module for basic hemodialysis, and a therapy module.

In an example, the home hemodialysis machine implemented for heavy drinking water may include a water module for advanced water purification, a dialysate module for basic hemodialysis, and a therapy module.

In an example, an in-center hemodialysis with central dialysis distribution system may include a water module for purified water, a dialysate module for basic hemodialysis, and a therapy module.

In an example, an in-center hemodialysis with heavy drinking water may include a water module for purified water, a dialysate module for basic hemodialysis, and a therapy module.

In an example, an acute hemodialysis with bagged fluid may include a water module for bagged dialysate, a dialysate module for advanced hemodialysis, and a therapy module.

In an example, an acute hemodialysis with standard drinking water may include a water module for standard water purification, a dialysate module for advanced hemodialysis, and a therapy module.

The system described herein may provide a single common user interface for all forms of dialysis, reducing training and service requirements. Other exemplary aspects of the system include the ability to change the purpose of a machine without changing the user interface. Still further exemplary aspects may include the ability to swap a portion of the system if it malfunctions, rather than switching the entire unit. The modules are sufficiently small to ship with standard shipping companies and not require a pallet shipment. Still further exemplary aspects may include the ability to have the exact same treatment from the same machine in a home setting as a patient does in the clinic. Still further exemplary aspects may include the ability to change the water source of a hemodialysis machine without changing the rest of the machine.

With reference to the following figures, an example integrated hemodialysis machine is described in detail first, and then additional components of an example active carbon dialysate filter hemodialysis machine and an example universal active carbon dialysate filter machine are described.

Integrated Hemodialysis Machine.

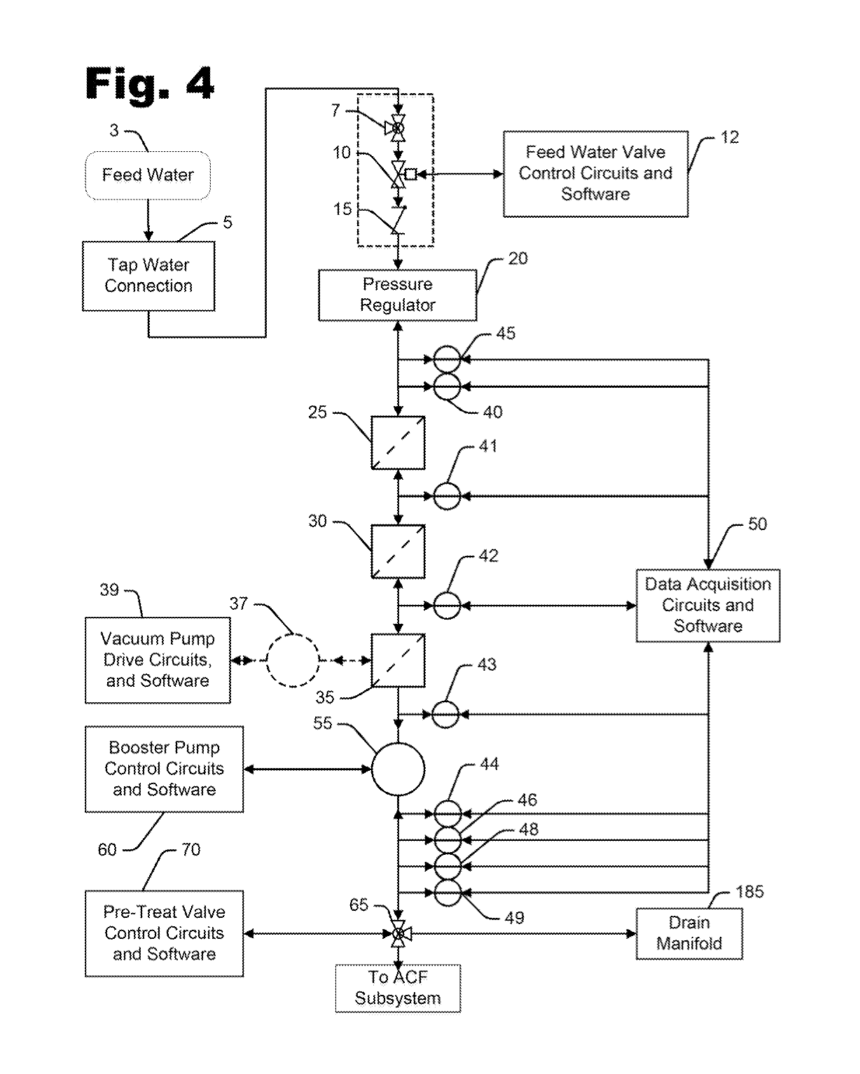

FIG. 4 is an illustration of an example pre-treatment subsystem. The pre-treatment subsystem receives feed water and pre-filters it in preparation for subsequent filtration steps. In an example, system components include (optional) Pre-Treatment Fitters, (optional) Booster Pump, Feed Water 3, Tap Water Connection 5, Manual Three-Way Valve 7, Feed Water Valve 10, Feed Water Valve Control Circuits and Software 12, Backflow Prevention Valve 15. Pressure Regulator 20 (e.g., to set pressure greater than 10 psi and less than 150 psi), Sediment Filter 25 (e.g., having a pore size less than or equal to 10 microns), Ultrafilter 30 (e.g., having a pore size less than or equal to 0.22 microns) which is replaceable at regular intervals or when needed, Air Removal Filter 35, Vacuum Pump 37 (e.g., vacuum level greater than or equal to 5.0 inches of mercury), Vacuum Pump Control Circuits and Software 39, Pressure Sensors 40, 41, 42, 43, 44 (e.g., pressure range 0-150 pounds per square inch, and a resolution at least ±1 pounds per square inch), Flow Sensors 45, 46 (e.g., having flow range 0-5 liters per minute, and resolution at least ±0.1 liters per minute), Air In Line Sensor 48 (e.g., range 1-1000 microliter bubbles and resolution less than or equal to 5 microliters), Conductivity Sensor 49 (e.g., conductivity range 0-2000 micro Siemens per centimeter, and resolution less than or equal to ±5 micro Siemen per centimeter), Data Acquisition Circuits and Software 50, Booster Pump 55 (e.g., pressure head greater than 50 pounds per square inch, and flow rate greater than 5 liters per minute). Booster Pump Control Circuits and Software 60. Pre-Treat Valve 65, Pre-Treat Way Valve Control Circuits and Software 70, and Drain Manifold 185.

The Tap Water Connection 5 connects the device to a tap water source which seals the fluid path and introduces feed water 3 to the device. If the feed water 3 does not meet the EPA Primary Drinking Water requirements, then pre-treatment filters may be implemented to reduce contaminants in the water.

A manual three way valve 7 is placed after the tap water connection to allow the user to shut-off or divert water flow to the device during installation, maintenance, or safety reasons.

Feed water then flows through the automated feed water valve 10 which allows the machine to control the ingress of feed water. This may be closed during installation, maintenance, or when flow is not required. The valve is controlled and driven by the feed water control circuits and software 12.

The feed water enters a backflow prevention valve 15 which keeps fluid in the system from flowing back in to the feed water supply.

The feed water then flows through a pressure regulator 20, which prevents over-pressure in the system. If the feed water is not sufficiently pressurized, a booster pump may be implemented in the feed water system before the tap water connection 5 to increase pressure.

A flow sensor 45 is located after the pressure regulator to determine the flow of the feed water. The sensor output is captured by the data acquisition circuits and software 50.

A pressure transducer 40 is in fluid communication with the fluid path after the regulator to monitor the inlet feed water pressure. The sensor output is captured by the data acquisition circuits and software 50.

The feed water then enters a sediment fitter 25 with pore size less than or equal to 10 micron. This filter reduces chemical and biological contaminants and prevents large particles from fouling downstream filters. The sediment filter is replaceable at regular intervals or when necessary.

A pressure transducer 41 is located after the sediment filter. The pressure drop through the sediment filter, as referenced to the pressure transducer 40, is monitored over time to determine the life of the filter. Fouling in the filter may cause the pressure drop to increase. The sensor output is captured by the data acquisition circuits and software 50.

The feed water then flows through an ultrafilter 30 with pore size less than or equal to 0.2 micron. The filter removes bacteria, virus, and endotoxin from the feed water. The ultrafilter is replaceable at regular intervals or when necessary.

A pressure transducer 42 is in fluid communication with the feed water after the ultrafilter 30. The pressure drop across the ultrafilter, as referenced to the pressure transducer 41, is monitored over time to determine the life of the filter. The sensor output is captured by the data acquisition circuits and software 50.

The feed water then flows through an air removal filter 35 to remove air and gas. The air removal filter is replaceable at regular intervals or when necessary.

A vacuum pump 37 may optionally be attached to the air removal filter to enhance air removal by drawing a vacuum in the fitter. The vacuum pump is driven and controlled by the vacuum pump drive circuits and software 39.

A pressure transducer 43 is in fluid communication with the feed water after the air removal filter 35. The pressure drop across the air removal filter, as referenced to the pressure transducer 42, is monitored over time to determine the life of the filter. The sensor output is captured by the data acquisition circuits and software 50.

The feed water then enters a booster pump 55, which may be positive displacement or centrifugal pump, in order to increase the pressure of the fluid stream after the pressure drops in the degassing, sediment, and ultra-filters. The booster pump is driven and controlled by the booster pump control circuits and software 60.

A pressure sensor 44 is located after the booster pump to determine the pressure of the water stream. The sensor output is captured by the data acquisition circuits and software 50.

A flow sensor 45 is in-line with the flow to determine the flow rate of feed water. The sensor output is captured by the data acquisition circuits and software 50.

An air in line sensor 48 measures the quantity of air in the water stream. The sensor output is captured by the data acquisition circuits and software 50.

A conductivity sensor 49 is located before the three way valve 65 in order to measure the ionic content in the water stream. The sensor output is captured by the data acquisition circuits and software 50.

The treated water then enters an automated three-way valve 65. The pre-treat valve 65 is driven and controlled by the valve control circuits and software 70.

The flow 46 and pressure 44 sensors provides feedback for the booster pump. If flow or pressure is too low, the booster pump input may be increased. If the flow or pressure is too high, the booster pump input may be decreased.

If the processor determines the water needs to be diverted to drain, the valve is activated and flow is diverted to the drain manifold 185. Otherwise, the water flows to the Active Carbon Filter Subsystem.

The system may be put in an installation or maintenance mode where water is diverted to the drain manifold 185 by the pre-treat valve 65 for a predetermined period of time or until it meets certain pressure, flow, or air requirements.

In an example, an additional booster pump may be implemented before entering the tap water connection 5. In the event the feed water pressure is low, a booster pump may be required to get adequate flow through the Pre-Treatment Subsystem.

In an example, additional filtering pre-treatment may be provided before entering the tap water connection. If the water has a high presence of a particular chemical or particulate, additional pre-treatment before the system may be necessary.

In an example, the air removal filter 35 and/or vacuum pump 37 may not be used, and may be an optional pre-treatment.

In an example, a shutoff valve (e.g., instead of the three way valve 65 and drain connection 185) may be provided at the end of the subsystem.

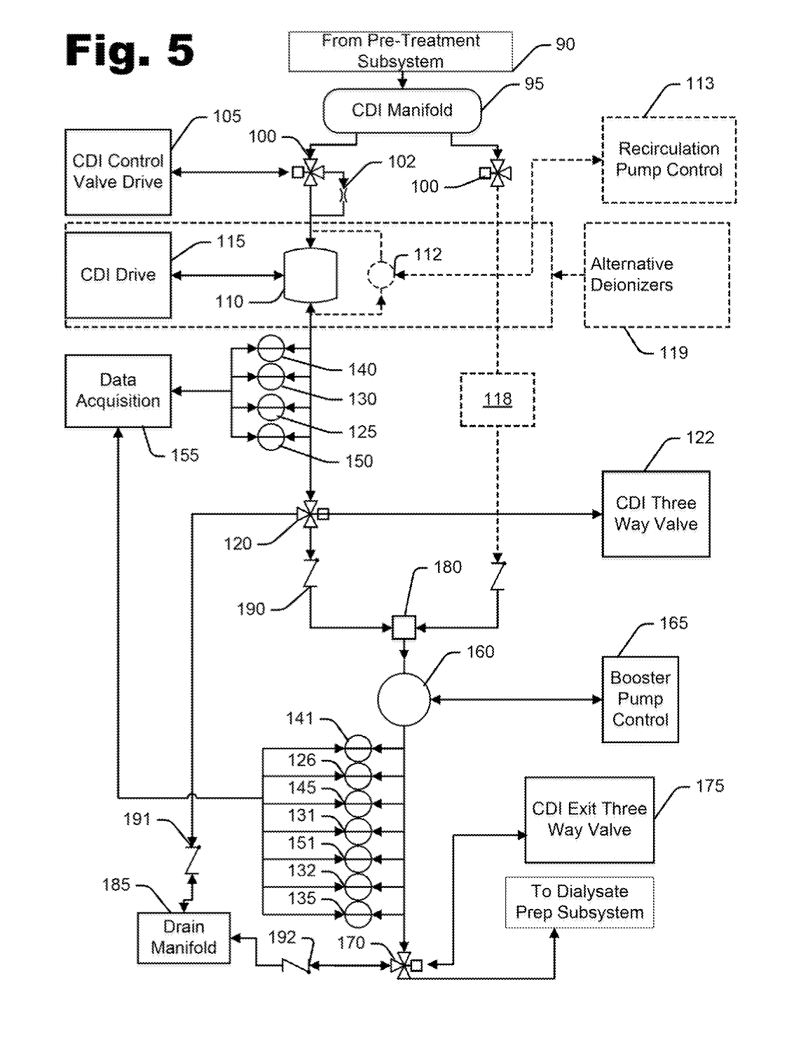

FIG. 5 is an illustration of an example Active Carbon Filter (ACF) Subsystem. The active carbon filter subsystem receives the output of the Pre-Treatment Subsystem and then removes ions and chemicals from the water.

In an example, the active carbon filter subsystem receives Water from Pre-Treatment Subsystem 90, and includes CDI Manifold 95, CDI Control Valves 100, CDI Flow Restrictor 102 (e.g., flow rate reduced least 50% when compared to high flow fluid path), CDI Control Valve Drive Circuits and Software 105, CDI Chamber 110 (e.g., flow rate at least 250 milliliters per minute while purifying to 1.0 micro Siemens per centimeter), Recirculation Pump 112, Recirculation Pump Control Circuits and Software 113, CDI Drive Circuits and Software 115, Additional CDI Fluid Circuits 118, Alternative Deionizers 119, CDI Three Way Valve(s) 120, CDI Three Way Valve Drive Circuits and Software 122, Pressure Sensors 125, 126 (e.g., pressure range 0-150 pounds per square inch, and resolution less than or equal to ±1 pounds per square inch), Conductivity Sensors 130, 131 (e.g., conductivity range at least 0-200 micro Siemens per centimeter, resolution less than or equal to ±0.25 micro Siemens per centimeter), Total Chlorine Sensor 132 (e.g., range 0-20 parts per million, and resolution less than or equal to 0.1 parts per million), Free Chlorine Sensor 135 (e.g., range 0-20 parts per million, and resolution less than or equal to 0.1 parts per million). Flow Sensors 140, 141 (e.g., flow range 0-5 liters per minute, and resolution less than or equal to ±0.1 liters per minute), pH Sensor 145 (e.g., pH range 0-14 pH units, and resolution less than or equal to ±0.25 pH units), Temperature Sensor(s) 150, 151 (e.g., temperature range 0-100° C., and resolution less than or equal to ±1° C.), Data Acquisition Circuits and Software 155, Booster Pump 160 (e.g., pressure head greater than 50 pounds per square inch, and flow rate greater than 5 liters per minute), Booster Pump Control Circuits and Software 165, CDI Exit Three Way Valve 170, CDI Exit Valve Control Software 175, Deionized Water Manifold 180, Drain Manifold 185, and Check Valves 190, 191, 192.

The pre-treated water 90 enters the ACF Subsystem from the Pre-Treatment Subsystem. The pre-treated water enters the CDI manifold 95 that divides the flow in to one or more fluid paths. One fluid path is demonstrated in the figure, but zero or more CDI fluid circuits 118 comprising the fluid circuit description below may be added.

The CDI fluid circuit is described as follows. Fluid enters the CDI control valve 100. The control valve is automated and has three states: no flow, low flow, or high flow. The CDI control valve drive circuits and control software 105 control the state of the valve and drive its movement. In the no flow state, the valve blocks flow to the remainder of the CDI fluid circuit. This may occur when the machine is not in use or during maintenance. In the low flow state the valve diverts flow through the CDI flow restrictor 102 and in to the CDI chamber 110. This may occur during regeneration of the CDI chambers. In the high flow state, the valve diverts flow directly to the CDI chamber 110. This may occur in a deionizing mode, maintenance, filter replacement, or regeneration.

In the CDI chamber 110 the fluid is deionized. The deionization chambers are made from carbon material (activated carbon fiber, granular activated carbon, block granular activated carbon, aerogel, reticulated vitreous carbon, etc). Water flows through the electrodes. Two electrodes of opposite charge create an static electric field where ions and charged chemicals are attracted to either the positive or negative electrode. The charge is generated by connection with a DC power supply. A differential voltage of between 1.2 and 10 Volts is applied to the electrodes. The voltage may vary, depending on the position in the electrode stack. The electrodes are encased in a non-leaching polymer housing. The electrodes are spaced by a polymer mesh or spacer. The spacing between electrodes may vary with position in the stack.

The CDI chamber voltage is driven and controlled by the CDI drive circuits and software 115. Once the fluid leaves the CDI chamber, four sensors monitor physical properties of the water. Pressure 125, Conductivity 130, Temperature 150, and Flow 140. The sensor output is captured by the data acquisition circuits and software 155. After the sensors, the CDI three way valve 120 diverts flow between two paths. The first path is through a check valve 191 and in to the drain manifold 185. The check valve prevents fluid intended for disposal from re-entering the CDI fluid circuit. The second path is through a check valve 190 and in to the deionized water manifold 180, where water continues through the fluid circuit of the Stationary Filter system to the Mixing Subsystem. The check valve prevents water from another CDI fluid circuit from entering the fluid circuit.

After the deionized water manifold 180, the water passes through a booster pump 160. The Booster Pump Control Circuits and Software 165 drive the Booster Pump. The booster pump is intended to increase the pressure of the fluid in the system after the CDI Chambers.

The deionized water then enters the following sensors: Flow Sensor 141 to determine the flow of the water; Pressure Sensor 126 to determine the pressure of the water, pH Sensor 145 to determine the acidity/alkalinity of the water, Conductivity Sensor 131 to determine the ion/chemical content and conductivity of the water; Temperature Sensor 151 to determine the temperature of the water, Total Chlorine Sensor 132 to determine the total amount of chlorine in the water; and Free Chlorine Sensor 135 to determine the amount of free chlorine in the water.

The data acquisition circuits and software 155 convert the data from analog to digital and communicates the measurements to the processor. The CDI exit three way valve 170 diverts flow between two paths. The first path is through a check valve to the drain manifold 185. The check valve prevents fluid intended for disposal from re-entering the CDI fluid circuit. The second path is to the Mixing Subsystem.

The CDI exit three way valve 170 is controlled by the CDI exit three way valve control circuits and software 175. The CDI control valve 100 diverts water through either the high or low flow fluid paths, depending on the needs of the system. The CDI drive circuits and software 115 provide power to the electrodes. This may be in sequential steps or all at once.

Based on the measurements from the deionized water output sensors 125, 130, 140, 150, the power to the CDI chamber drive may be manipulated by the CDI drive circuits and software 115.

Until the deionized water meets specification, as determined by the data from the sensors 125, 130, 140, 150, the CDI three way valve 120 diverts flow to the drain manifold 185. Once the deionized water meets specification, as determined by the data from the sensors 125, 130, 140, 150, the CDI three way valve 120 diverts flow to the deionized water manifold 180. If at any point the water does not meet specification, then the CDI three way valve 120 is switched back to the drain manifold exit position.

Once the electrodes reach capacity (electrode surface is covered so that no more ions/chemicals may attach) and the specification is no longer being met, the CDI three way valve 120 immediately diverts the flow to the drain manifold 185 and the CDI chamber 110 goes in to regeneration mode.

During regeneration mode, the CDI drive circuits and software 115 manipulate power to the electrodes causing ions/chemicals to desorb from the electrode surfaces. The waste flow is diverted to the drain manifold 185 by the CDI three way valve 120.

During regeneration, the flow path may be diverted by the CDI control valve 100 to the high flow or low flow 102 circuits, depending on the regeneration needs.

Once regeneration criteria have been met (may be electrode current, electrode voltage, conductivity of effluent, etc), the system may either go in to a rest mode (no flow) or return to a purification mode.

In an example, there may be one or multiple CDI fluid circuits, drive circuits, and software 118. An accumulator tank may also be added in the CDI circuit. For example, a tank of <100 liter volume may be filled with sterilized feed water (from the Pre-Treatment subsystem). The CDI circuit may draw from the tank, deionize the water, and circulate it back in to the tank until the conductivity, chlorine, chloramine, and pH of the bulk fluid is at an acceptable level. The fluid may then be pumped in to the Deionized Water Manifold.

In an example, the CDI chamber may be replaced by an alternative deionizing system 119. including by way of example, Reverse Osmosis, Electro dialysis, Charged Ion Beds, Electrodeionization.

In an example, the CDI Chamber Electrode configuration may be flow-between electrodes instead of flow-through electrodes.

In an example, the electrodes in the CDI chamber 110 may have current passed across each one, rather than from one electrode to the next across the gap (as a capacitor). The internal resistance of the electrode causes it to heat up. When fluid flowed through the electrode, its temperature may raise due to convective heat transfer from the electrode. Heating fluid above a certain temperature threshold may allow for disinfection of the system and more effective regeneration. Electrodes may be used to heat fluid during purification to create a sterile solution at a specified temperature.

In an example, an optional recirculation pump 112 takes fluid from the output of the CDI chamber 110 and pumps it back in to the entrance of the CDI chamber. This recirculation has multiple purposes and functions. For example, the recirculating a portion the fluid allows the fluid to pass through the CDI chamber multiple times, effectively increasing the number of electrodes through which the fluid passes and therefore removing more charged particles (when deionizing), increasing the temperature (when heating), or increasing the concentration of waste particles in the stream (if regenerating). The recirculated fluid mixed with the influent effectively mixes the properties of the two streams.

At times, it may be desirable to simply recirculate the fluid from the outlet of the CDI chamber back in to the inlet. This may be used in a “standby mode”—waiting for the user to begin using the system to create a batch of fluid or an on-demand stream of fluid.

In an example, the flow rate of recirculation pump may be variable from 0 to 1000% of the influent flow rate.

In an example, the recirculation pump 112 is controlled and driven by the recirculation pump control circuits and software 113.

In an example, a two or three way valve may be used to connect the entrance and exit of the recirculation pump to the inlet and outlet streams of the CDI chamber 110.

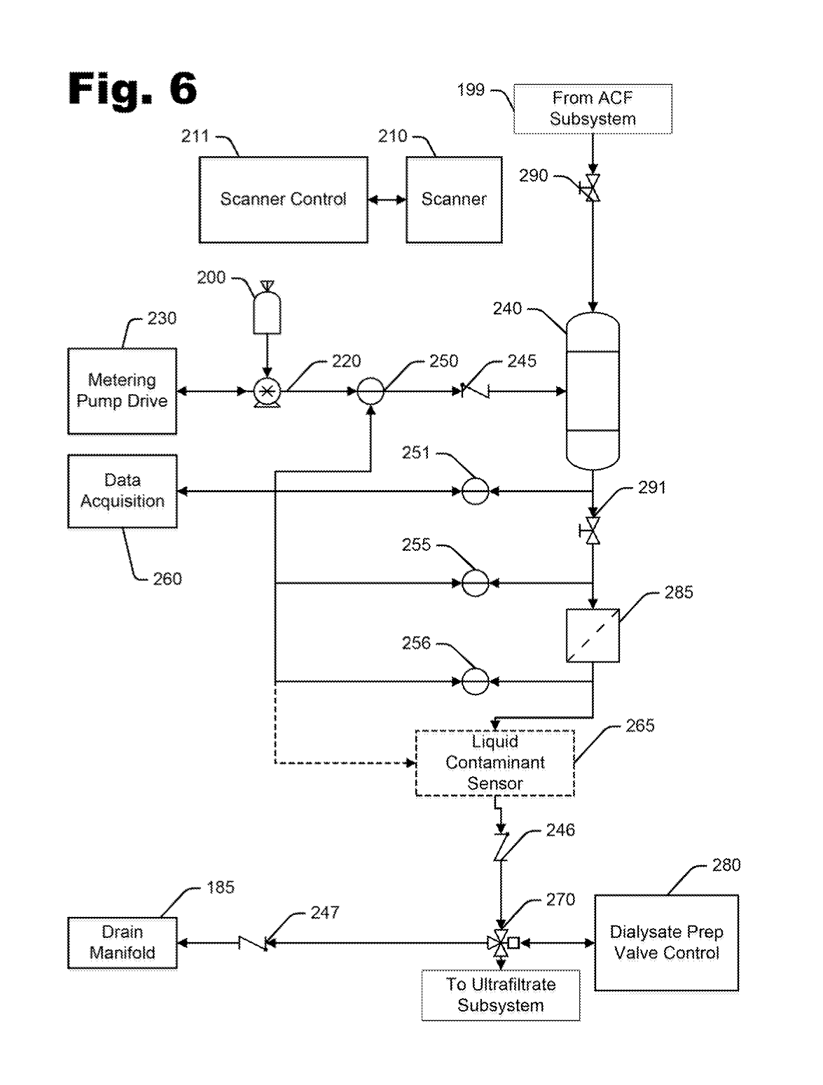

FIG. 6 is an illustration of an example dialysate preparation subsystem. The dialysate preparation subsystem combines concentrate with deionized water to form a fluid of specified concentration and then removes bacteria, virus, and endotoxin from the solution by ultrafiltration.

In an example, the dialysate preparation subsystem includes a Drain Manifold 185, Water from ACF Subsystem 199, One or more containers of concentrate solution 200 (e.g., volume greater than 500 milliliters), Barcode Scanner 210, Barcode Scanner Control Circuits and Software 211, Metering Pump 220 (e.g., having a flow rate range 0-100 milliliters per minute, pressure head greater than or equal to 50 pounds per square inch). Metering Pump Control Circuits and Software 230, Mixing Chamber 240, Check Valves 245, 246, 247, Conductivity Sensor 250, 251 (e.g., conductivity Range 0-40,000 micro Siemens per centimeter, and a resolution less than or equal to 100 micro Siemens per centimeter), Pressure Sensors 255, 256 (e.g., pressure range 0-150 pounds per square inch, and resolution less than or equal to +1 pounds per square inch), Data Acquisition Circuits and Software 260, Liquid Contaminant Sensor 265 (e.g., capable of detecting and/or counting particles <30 micron), Dialysate Prep Valve 270, Dialysate Prep Valve Control Circuits and Software 280, Ultrafilter 285 (e.g., having a pore size less than or equal to 0.22 microns), and Sample Port 290, 291

Water (dilutant) from the ACF Subsystem enters the Dialysate Preparation Subsystem 199. The fluid from the ACF Subsystem passes through a sample port 290. The sample port 290 may be a push button port with a spout the can be easily accessed for filling a sample container. This is for capturing samples of fluid for required water quality tests. The dilutant enters the mixing chamber 240. The mixing chamber may be a separate volume or simply a stretch of tubing.

A barcode scanner 210 (or the like) that may be convenient to the user, is employed to read critical information from the concentrate solution labeling. Critical information may include solution description, concentration, lot number, or expiration date. The bar code scanner is controlled by the bar code scanner control circuits and software 211.

Concentrate is added to the mixing chamber by one or more of the concentrate circuits. Each concentrate circuit may include the following.

One or more containers of medical solution concentrate 200 are connected to the metering pump 220. The concentrate may be of injection, irrigation, hemodialysis, hemofiltration, hemodiafiltration, or other medical fluids.

The metering pump 220 apportions the concentrate solution 200 in to the mixing chamber 240.

The metering pump 220 is driven and controlled by the metering pump control circuits and software 230.

The concentrate exits the metering pump 220 and enters a conductivity sensor 250. The conductivity sensor 250 checks that the conductivity of the fluid matches what is expected to be in the system. Data acquisition circuits and software 260 capture the conductivity measurement from the sensor and store the value in memory for access by the system software.

After the conductivity sensor 250 the concentrate enters a check valve 245. The check valve 245 prevents concentrate or dilutant from flowing back in to the metering pump 220 and concentrate container 200.

After exiting the check valve 245, the concentrate enters the mixing chamber 240.

The concentrate and dilutant enter the mixing chamber 240 where the solutions are mixed.

After exiting the mixing chamber 240, the fluid passes a conductivity sensor 251 for a check of the solution's conductivity against known values for the desired solution and concentrate. Data acquisition circuits and software 260 capture the conductivity measurement from the sensor and store the value in memory for access by the system software.

The fluid passes through a sample port 291. The sample port 291 may be a push button port with a spout the can be easily accessed for filling a sample container. This is for capturing samples of fluid for required water quality tests.

After exiting the sample port 291, a pressure sensor 255 monitors the pressure of the fluid. The sensor output is captured by the data acquisition circuits and software 260.

After exiting the pressure sensor 270, the solution enters the ultrafilter 285. which removes bacteria, virus, and endotoxin. The size of the ultrafilter pores is less than 0.22 micron.

After exiting the ultrafilter 285, the pressure of the fluid is measured by a pressure sensor 256. The sensor output is captured by the data acquisition circuits and software 260. The pressure drop through the ultrafilter, as referenced to the pressure sensor 255 before the ultrafilter 285. is monitored over time to determine the life of the filter. Fouling in the filter may cause the pressure difference between the two sensors to increase. The sensor output is captured by the data acquisition circuits and software 260.

The solution passes the pressure sensor 256 and enters a check valve 246, which prevents fluid from being pushed back in to the Dialysate Preparation Subsystem.

The fluid exits the check valve 246 and enters a three way valve, the dialysate prep valve 270. The three way valve 270 has two positions: the first directs the flow to the drain manifold 185, and the second directs the flow to the Ultrafiltrate Subsystem.

After exiting the dialysate prep valve 270, the solution passes through a check valve 247 before entering the drain manifold 185. This valve prevents fluid intended for disposal from re-entering the subsystem.

The system software communicates with the flow sensors in the ACF subsystem 140 or 141 to determine the correct flow rate of concentrate 200, and by calculation the speed of the metering pump 220. The flow rate of the concentrate solution is based on the measured flow rate of the dilutant, the desired concentration of solution, and the concentration of the concentrate (from the barcode scanner software). The concentration may also be controlled with feedback from the conductivity sensor 251.

The system software reads the critical information from the barcode scanner 210 and software 211. This data is logged and used to determine the appropriate concentration of fluid, and therefore the speed of the metering pump.

The conductivity of the solution, as measured by the conductivity sensor 251, is compared against known values for the desired concentration of solution. If the conductivity is in the acceptable range, the dialysate preparation valve 270 allows the fluid to continue to the Output Subsystem. If the fluid fails the conductivity test, the dialysate preparation valve 270 directs the fluid to the drain manifold 185. If the fluid is found unacceptable for a significant period of time, the user is alerted. The default position of the mixing valve 270 is open to the drain manifold 185.

If the conductivity of the concentrate, as measured by the conductivity sensor 250, is not consistent with the expected value, then the user is alerted and the metering pump 220 stops apportioning the concentrate.

In an example, any sort of optical or RFID recognition of the concentrate solution instead of a barcode scanner 210, 211. This may include QR codes, 1D barcodes, visual symbols by image detection, etc.

In an example, this subsystem may be omitted from the system, e.g., if the purpose of the machine is solely to generate sterile water.

In an example, the check valve 245. concentrate manifold 248, and mixing chamber 240 may be one integrated physical unit.

In an example, addition of a booster pump before the ultrafilter in order to boost pressure and flow through the filter and then the Output Subsystem.

In an example, a liquid contaminant sensor 265 may be present after the ultrafilter 285 in order to measure contaminants (including ions, molecules, particles, endotoxin, virus, or bacteria). The measurement of contaminants may be used to determine the suitability of the water quality based on magnitude of contaminants in the product water stream. If the particle sensor 265 detects particles greater than a predetermined threshold of acceptability, the dialysate prep valve 270 diverts the solution to the drain manifold 185 and the user is alerted.

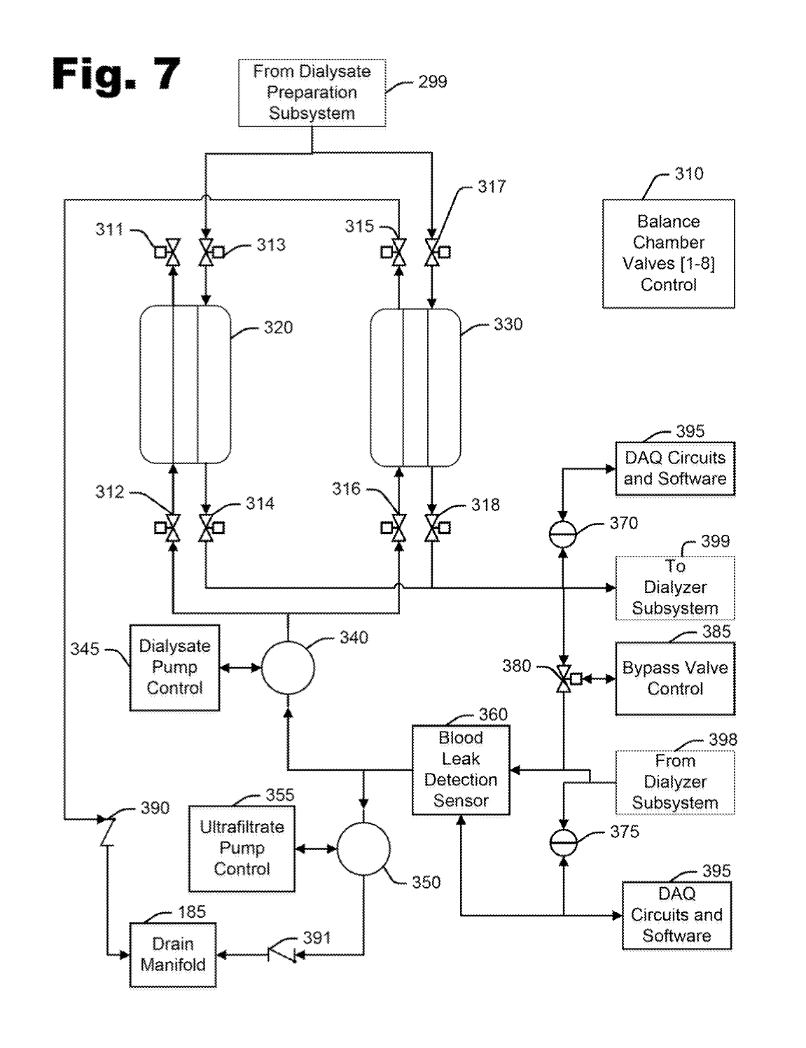

FIG. 7 is an illustration of an example Ultrafiltrate Subsystem. The Ultrafiltrate Subsystem manages dialysate fluid flow to and from the dialyzer.

The Ultrafiltrate Subsystem receives Solution from Dialysate Preparation Subsystem 299. In an example, the Ultrafiltrate Subsystem includes Balance Chamber Valves 1-8 Control Circuits and Software 310, Balance Chamber Valve 1 311, Balance Chamber Valve 2 312, Balance Chamber Valve 3 313, Balance Chamber Valve 4 314, Balance Chamber Valve 5 315, Balance Chamber Valve 6 316, Balance Chamber Valve 7 317, Balance Chamber Valve 8 318, Balance Chamber 1 320, Balance Chamber 2 330, Dialysate Pump 340, Dialysate Pump Control Circuits and Software 345, Ultrafiltrate Pump 350, Ultrafiltrate Pump Control Circuits and Software 355. Blood Leak Detection Sensor 360, Fresh Dialysate Dialyzer Pressure Sensor 370, Spent Dialysate Dialyzer Pressure Sensor 375, Bypass Valve 380. Bypass Valve Control Circuits and Software 385, Check Valves 390, 391, DAQ Circuits and Software 395, Spent Dialysate from Dialyzer Subsystem 398, Fresh Dialysate to Dialyzer Subsystem 399, and Drain Manifold 185.

Dialysate solution enters the subsystem from the Dialysate Preparation Subsystem 299. The fresh dialysate is split between the two balance chambers 320, 330. The dialysate enters through balance chamber valve 3 313 and balance chamber valve 7 317. The position of all eight of the balance chamber valves 311-318 are controlled by the balance chamber valve control circuits and software 310. Fresh dialysate exits the balance chambers 320, 330 through balance chamber valve 4 314 and balance chamber valve 8 318.

After exiting the balance chamber valves, the pressure of the fresh dialysate is measured by the fresh dialysate dialyzer pressure sensor 370. The sensor output is captured by the data acquisition circuits and software 395. The fresh dialysate then enters the Dialyzer Subsystem 399.

Spent dialysate is received from the Dialyzer Subsystem 398. The spent dialysate pressure is measured by the spent dialysate dialyzer pressure sensor 375. The sensor output is captured by the data acquisition circuits and software 395.

After the pressure sensor 375, the spent dialysate passes through a blood leak detection sensor 360. This sensor looks for red blood cells in the spent dialysate, which indicate a broken fiber in the dialyzer. The output of the blood leak detection sensor 360 is captured by the data acquisition circuits and software 395.

After the blood leak detection sensor 360, the spent dialysate flows in to two pumps, the dialysate pump 340 and the ultrafiltrate pump 350. The dialysate pump 340 pumps spent dialysate in to the balance chambers 320, 330. The ultrafiltrate pump 350 pumps a regulated amount of spent dialysate (ultrafiltrate) through a check valve 391 and in to the drain manifold 185. This ultrafiltrate is the fluid taken off the patient through hemodialysis.

The dialysate pump 340 is controlled and driven by the dialysate pump control circuits and software 345. The ultrafiltrate pump 350 is controlled and driven by the ultrafiltrate pump control circuits and software 355.

The spent dialysate that is returned to the balance chambers 320, 330 enters through balance chamber valve 2 312 and balance chamber valve 6 316.

The spent dialysate leaves the balance chambers 320, 330 through balance chamber valve 1 311 and balance chamber valve 5 315. After exiting the balance chamber valves, the spent dialysate flows through a check valve 390 and in to the drain manifold 185. The check valve 390 prevents fluid from the drain manifold entering the Ultrafiltrate Subsystem.

The bypass valve 380 allows fresh dialysate to bypass the Dialyzer Subsystem and flow directly in to the spent dialysate loop. The bypass valve 380 is controlled and driven by the bypass valve control circuits and software 385.

The balance chambers 320, 330 provide precise volumetric control of fluids entering and exiting the dialyzer. The chambers are a fixed control volume with a flexible membrane between the fresh and spent dialysate portions of the chamber. At any point in time, the exit valve of one side of the chamber (fresh dialysate or spent dialysate) is closed and the entrance is open, allowing the particular side to fill. On the other side of the flexible membrane, the entrance valve is closed and the exit valve is open allowing the fluid to exit. The pressure from the filling side pushes the fluid out of the emptying side. By using two chambers, the exiting and filling sides can be alternated and a constant flow of dialysate to and from the dialyzer can be achieved.

The pressure sensors on the dialyzer input 370 and output 375 assist in determining the transmembrane pressure, which is the pressure across the dialyzer membrane.

If the blood leak detection sensor 360 detects the presence of red blood cells in the spent dialysate, the software alerts the user and takes precautions with the flow of fluid through the system.

The volumetric flow of the ultrafiltrate pump 350 is controlled by the user or a predetermined figure. The ultrafiltrate flow determines the amount of waste product that is being taken out of the patient's blood.

It is noted that other methods for volume control may be employed in place of the balance chambers, including flow meters.

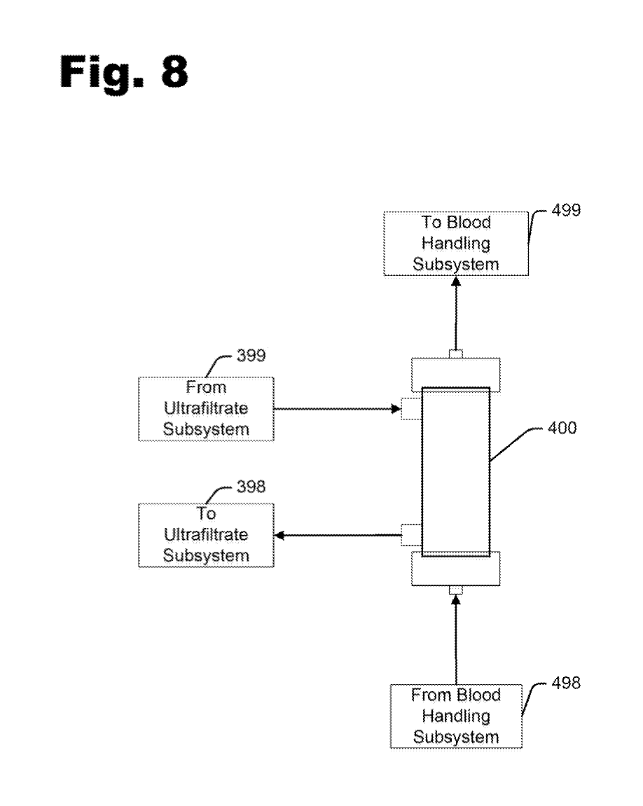

FIG. 8 is an illustration of an example Dialyzer Subsystem. The Dialyzer Subsystem provides the mechanism for exchanging dialysate and blood fluids in a controlled manner. The Dialyzer Subsystem may include Spent Dialysate to Ultrafiltrate Subsystem 398, Fresh Dialysate from Ultrafiltrate Subsystem 399, Hollow Fiber Hemodialyzer 400, Blood from the Blood Handling Subsystem 498, and Blood to the Blood Handling Subsystem 499.

The hollow fiber hemodialyzer 400 provides a dialyzing membrane across which blood toxins and/or other substances are exchanged.

The hemodialyzer 400 receives fresh dialysate 399 from the Ultrafiltrate Subsystem. The fresh dialysate 399 is run through the hemodialyzer 400 and by dialysis is infused with waste substances and fluid from the blood. The result is spent dialysate 398, which is returned to the Ultrafiltrate subsystem.

The hemodialyzer 400 receives arterial blood from the blood handling system 498. The blood 498 runs through the fibers of the hemodialyzer 400. While in the dialyzer the blood 398 loses waste substances and fluid. The blood exits the hemodialyzer as venous blood and is returned to the blood handling system 499.

In an example, another dialyzer technology, other than hollow fiber, may be used for dialysis, including: flat membrane dialyzer, microfluidic dialyzer, etc.

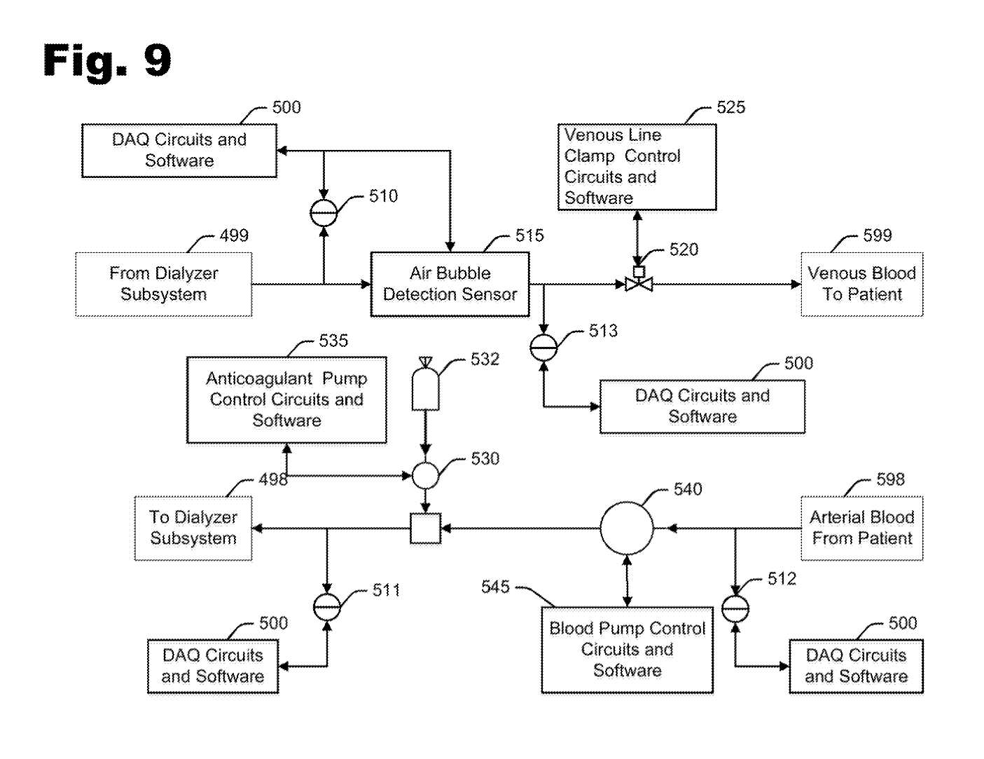

FIG. 9 is an illustration of an example Blood Handling Subsystem. The Blood Handling Subsystem takes blood from the patient, conducts it to the dialyzer, receives it from the dialyzer, and returns it to the patient. The Blood Handling Subsystem may include Arterial Blood to Dialyzer Subsystem 498, Venous Blood from Dialyzer Subsystem 499, DAQ Circuits and Software 500, Venous Dialyzer Pressure Sensor 510, Arterial Dialyzer Pressure Sensor 511, Arterial Blood Pressure Sensor 512, Venous Blood Pressure Sensor 513, Air Bubble Detection Sensor 515, Venous Line Clamp 520, Venous Line Clamp Control Circuits and Software 525, Anticoagulant Pump 530, Anticoagulant 532, Anticoagulant Pump Control Circuits and Software 535, Blood Pump 540, and Blood Pump Control Circuits and Software 545.

Arterial blood from the patient 598 enters the system through an intravenous connection with the patient's circulatory system. The pressure of the arterial blood from the patient 598 is measured by the arterial blood pressure sensor 512. The output of the arterial blood pressure sensor 512 is captured by the data acquisition circuits and software 500. The arterial blood pressure sensor 512 may be capable of measuring a negative line pressure.

After the arterial blood pressure sensor 512, the blood enters the blood pump 540. The blood pump 540 provides a motive force for pushing the blood through the dialyzer. The blood pump 540 is controlled and driven by the blood pump control circuits and software 545.

After the blood pump 540, the anticoagulant pump 530 injects anticoagulant 532 in to the blood. The anticoagulant 532 is used to prevent coagulation of blood in the dialyzer. The anticoagulant pump is controlled and driven by the anticoagulant pump control circuits and software 535. In an example, the anticoagulant may be heparin.

After anticoagulant has been added to the arterial blood, the pressure is measured. The arterial dialyzer pressure sensor 511 measures the pressure of the arterial blood before it enters the Dialyzer Subsystem 498. The output of the arterial dialyzer pressure sensor 511 is captured by the data acquisition circuits and software 500.

After the pressure sensor 511, the blood enters the Dialyzer Subsystem 498. Venous blood returns from the Dialyzer Subsystem 499 and passes a pressure sensor 510. The venous dialyzer pressure sensor 510 measures the pressure of the venous blood just after the dialyzer. The output of the venous dialyzer pressure sensor 510 is captured by the data acquisition circuits and software 500.

After the pressure sensor 510, the blood is monitored for the presence of air bubbles. The air bubble detection sensor 515 measures air bubbles in the venous blood. Air bubbles may cause stroke if returned to the circulatory system of the patient. The air bubble detection sensor 515 is the sensing part of the safety system that prevents air from returning to the patient's blood stream. The output of the air bubble detection sensor 515 is captured by the data acquisition circuits and software 500.

After the air bubble detection sensor 515, the pressure of the venous blood is measured by the venous blood pressure sensor 513. The output of the venous blood pressure sensor 513 is captured by the data acquisition circuits and software 500.

After the pressure sensor 513, the venous blood passes through the venous line clamp 520. The venous line clamp 520 is another component in the air bubble safety system. If an air bubble is detected by the air bubble detection sensor 515, then the venous line clamp 520 closes the blood line to the patient in order to prevent the air bubble from returning to the patient. The venous clamp 520 is controlled and driven by the venous line clamp control circuits and software 525. After the venous line clamp 520, the blood returns to the patient's circulatory system 599.

If an air bubble above a certain threshold size is detected in the venous blood by the air bubble detection sensor 515, then the venous clamp 520 is dosed to prevent the bubble from entering the patient's circulatory system.

Anticoagulant 532 is dosed in to the arterial blood stream prior to the Dialyzer Subsystem 498. The rate of blood coming from the blood pump 540 is used to appropriately does the anticoagulant using the anticoagulant pump 530 and its control circuits and software 535.

The speed of the blood pump 540 is controlled either by the user or specific predetermined sensors and is proportional to the rate of dialysate flow, as controlled by the dialysate pump 340 in the Ultrafiltrate Subsystem.

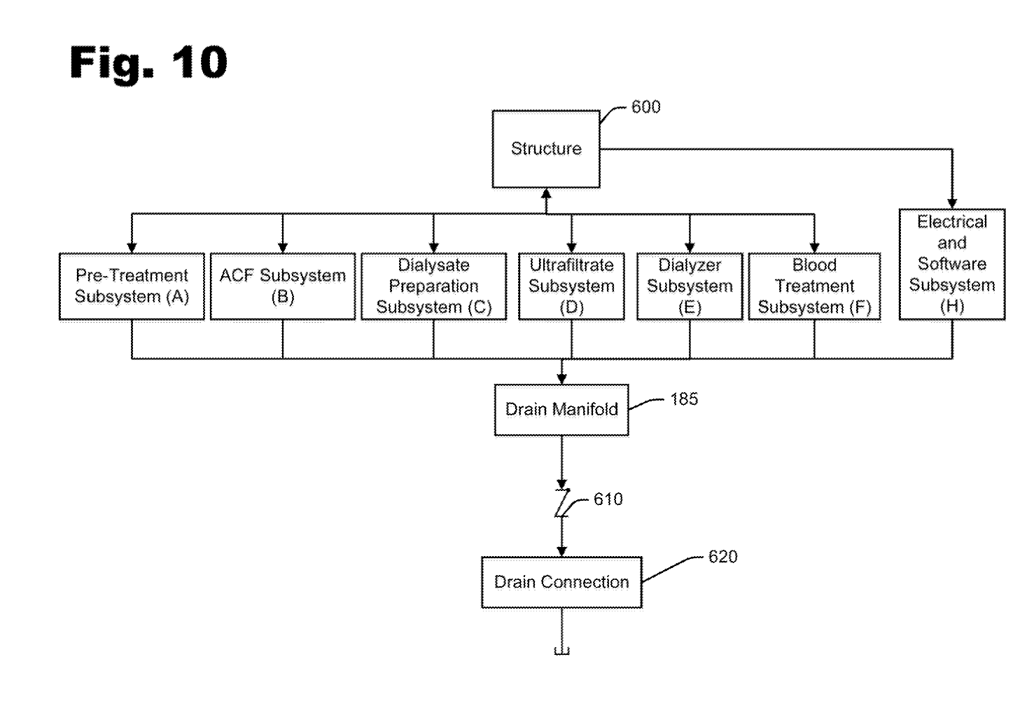

FIG. 10 is an illustration of an example Mechanical Subsystem. The Mechanical Subsystem provides a means for supporting all other subsystems. The Mechanical Subsystem may include Structure 600, Drain Manifold 185 (e.g., having a flow rate to drain at least two liters per minute), Check Valve 610, and Drain Connection 620.

The structure 600 allows points of connection to support the other subsystems in the Stationary Filter. The drain manifold 185 provides a point of connection for all the subsystems that direct fluid to the drain. It may include many inputs and one output to the check valve 610. The check valve prevents drain fluids from re-entering the system. The drain fluid exits the check valve 610 and enters the drain connection 620 where it is flushed in the drain.

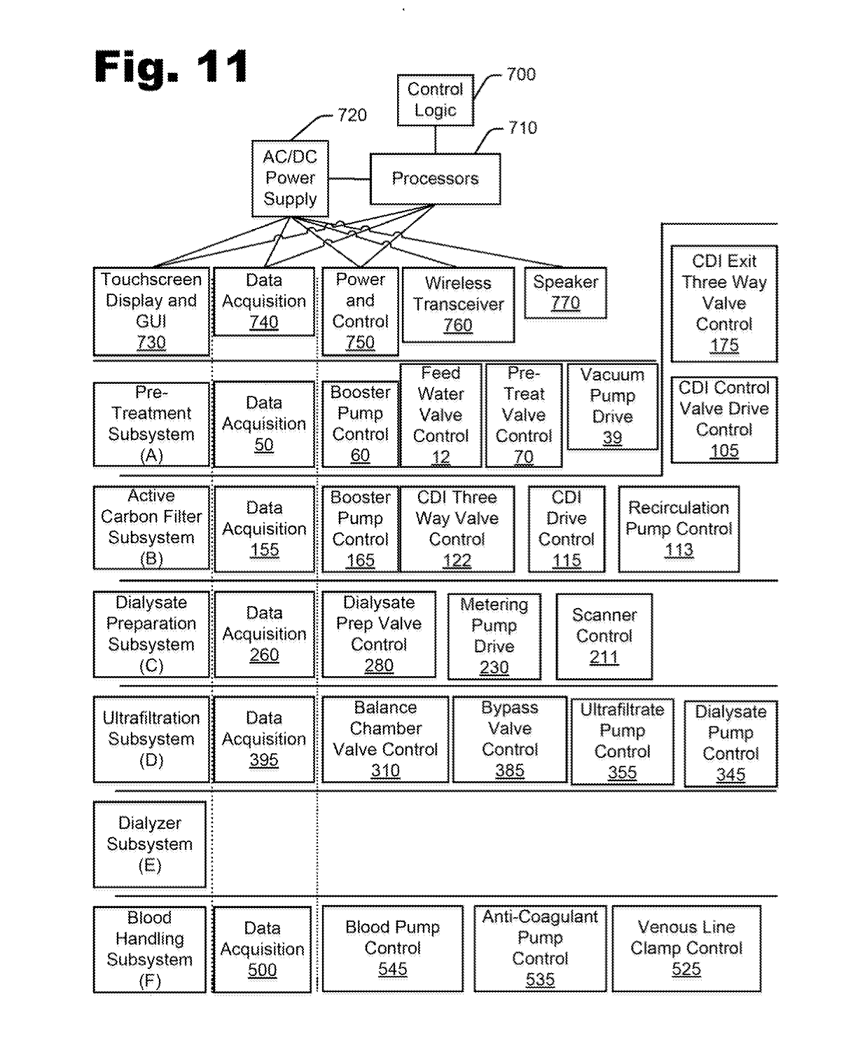

FIG. 11 is an illustration of an example Electrical and Software Subsystem. The Electrical and Software Subsystem provides a means for executing logic, acquiring data from mechatronic sensors, powering actuators, and providing a user interface. The Electrical and Software Subsystem may include Control Logic 700, Processors 710, AC/DC Power Supply 720, Touchscreen Display and Graphical User Interface 730, Data Acquisition Circuits 740, Power and Control Circuits 750, Wireless Transceiver 760, and Speaker 770.

AC power enters the AC/DC power supply 720. The power is converted from alternating current to direct current in a manner compliant with medical device standards.

The processors 710 are powered by the DC power from the power supply 720. The processors 710 execute the logic and safety measures required to run the device based on the control logic 700.

The data acquisition (DAQ) circuits 740 are powered by the power supply 720. The DAQ circuits 740 convert analog information from the sensors in the system to digital signals for the processors 710.

The touchscreen display 730 is powered by the power supply 720. It displays graphics as instructed by the processor 710 and control software 700 to create the graphical user interface. The touchscreen 730 communicates physical touch commands from the user to the processor 710. The control software 700 instructs the system based on the user commands.

The power and control circuits 750 are powered by the power supply 720. These circuits energize the actuators and control the position of the actuators as instructed by the processors 710.

The control logic 700 is a set of instructions that run on the processor 710.

The wireless communication transceiver 760 sends and receives data between the processor 710 and a local area network. It is powered by the power supply 720 and communicates information to and from the processor 710.

The speaker 770 is used for alerting the user and providing audible feedback, as determined by the control logic 700 and processor 710. The speaker 770 is powered by the power supply 720.

The pressure drop across (pressure before minus pressure after) a filter is known at a given flow rate. The system may measure the pressure drop across all filters (sediment filter, ultrafilter, air removal filter) and compare to known values. If the system observes that the pressure drop approaches the maximum acceptable value, the user may be notified that filter replacement may be required shortly. If the pressure drop exceeds the acceptable value, the hemodialysis system may stop functioning until the filters are replaced.

The device may be connected to the internet and send a signal to the manufacturer when the pressure drops approach the acceptable limit. This may trigger an automated reorder of the filter and shipment to the user.

The system may maintain usage data. This data may be stored on the hemodialysis machine, on an email account, on a local device via Ethernet, a server location, remote data storage via cell phone connection, or an EMR database. The data may include the following: Treatment details, including time- and treatment-stamped measurements for all sensors; Case details, including user information, patient information, treatment options selected, dialyzer used, concentrate used, sensor measurements, amount of fluid used, etc.; System errors, faults, and messages; and Maintenance Status, including filter life, total volume of fluid purified, number of treatments performed, etc.

The system may reorder concentrate as concentrate is used.

The hemodialysis machine may manage the pressure across the dialyzer membrane. By measuring the difference between the dialysate pressure (determined by dialyzer pressure sensors 370, 375) and the blood pressure (determined by the blood pressure sensors 510, 511), the hemodialysis machine can determine the transmembrane pressure in the dialyzer. That pressure may then be modified by increasing or decreasing the dialysate pump 340 and/or the blood pump 540.

Active Carbon Dialysate Filter Hemodialysis Machine.

An example of an active carbon dialysate filter hemodialysis machine is disclosed, including a pre-treatment subsystem; an active carbon filter subsystem; a dialysate preparation subsystem; an ultrafiltrate subsystem; a dialyzer subsystem; a blood handling subsystem; a mechanical subsystem; and an electrical and software subsystem.

In an example, the pretreatment subsystem, dialysate preparation subsystem, dialyzer subsystem, blood handling subsystem, mechanical subsystem, and electrical and software subsystem are as already described above for the integrated hemodialysis machine.

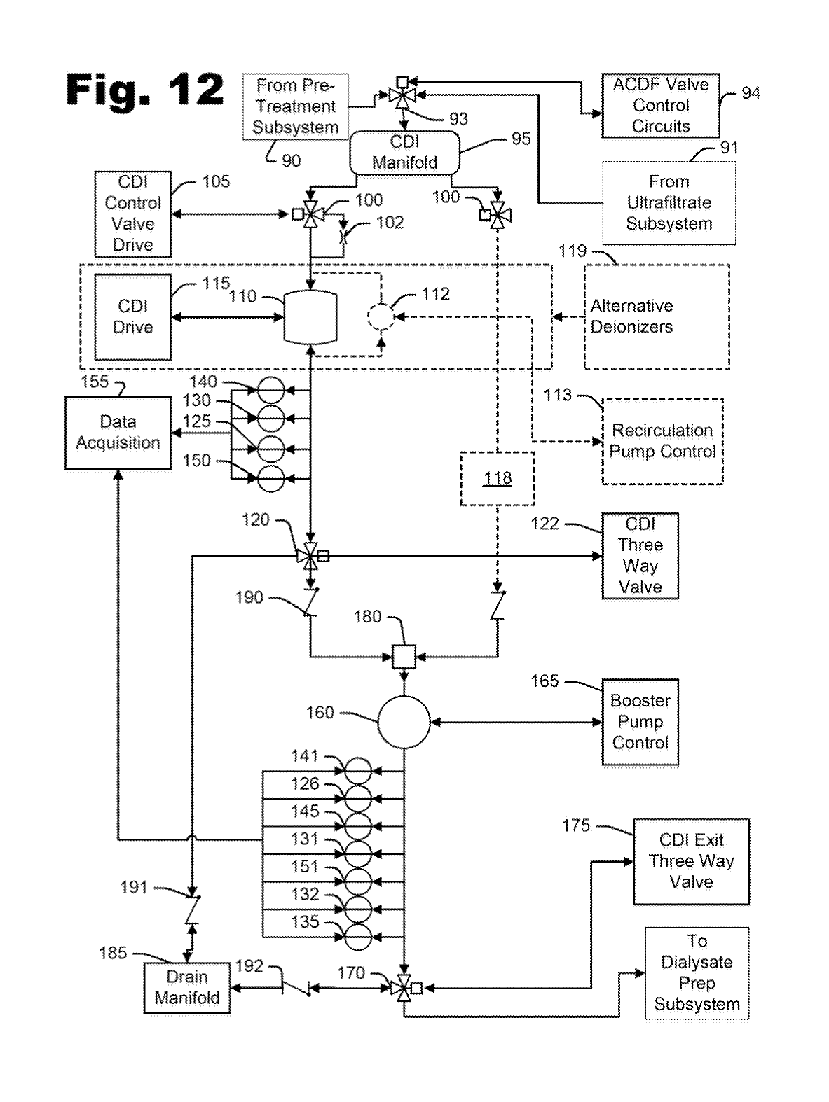

FIG. 12 shows an example active carbon dialysate filter subsystem of the active carbon dialysate filter hemodialysis machine. In an example, an active carbon filter subsystem includes a Spent Dialysate from Ultrafiltrate Subsystem 91; an Active Carbon Dialysate Filter (ACDF) Control Valve 93; and an ACDF Valve Control Circuits and Software 94 (e.g., a three way valve). Pre-treated water 90 enters the ACDF Subsystem from the Pre-Treatment Subsystem. Spent dialysate 91 enters the ACDF Subsystem from the Ultrafiltrate Subsystem. The ACDF Control Valve 93 is a three way valve that switches between one of two inputs—pre-treated water 94 and spent dialysate 91. The ACDF valve 93 is controlled and driven by the ACDF Valve Control Circuits and Software 94. The selected fluid enters the CDI manifold 95 that divides the flow in to one or more fluid paths. One fluid path is demonstrated in the figure, but zero or more CDI fluid circuits 118 comprising the fluid circuit description below may be added.

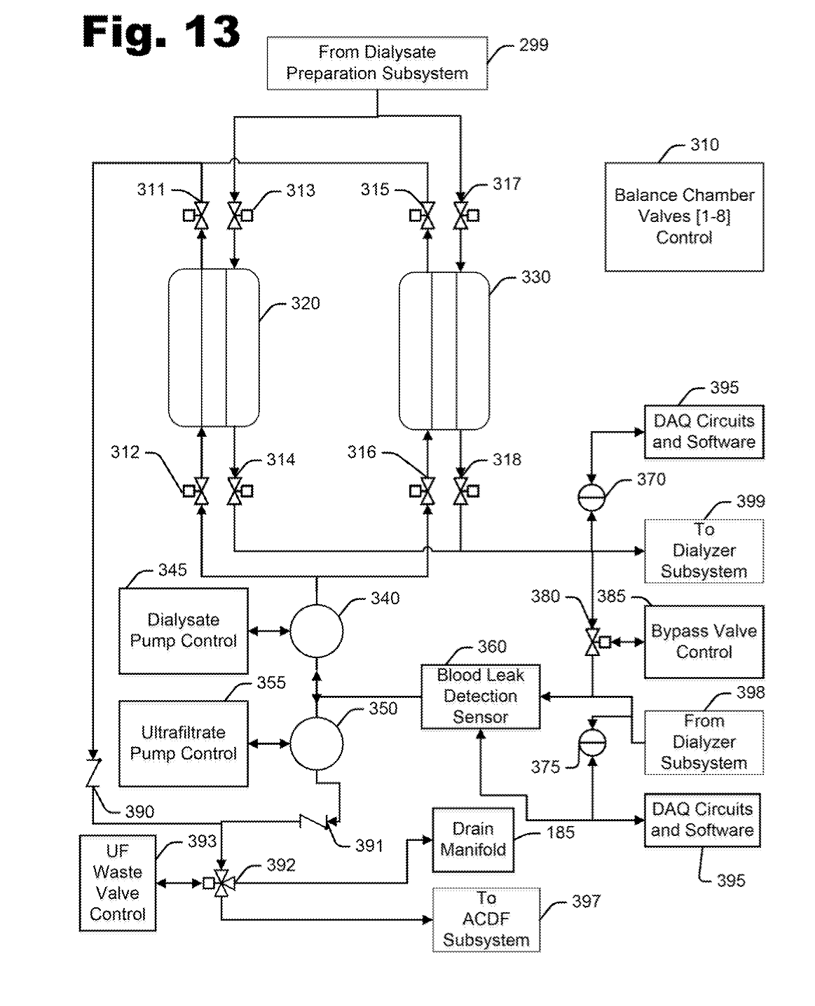

FIG. 13 shows an example ultrafiltrate subsystem of the active carbon dialysate filter hemodialysis machine. In an example, the ultrafiltrate subsystem includes UF Waste Valve 392, UF Waste Valve Control Circuits and Software 393, and Spent Dialysate to ACDF Subsystem 397. After the blood leak detection sensor 360, the spent dialysate flows in to two pumps, the dialysate pump 340 and the ultrafiltrate pump 350. The dialysate pump 340 pumps spent dialysate in to the balance chambers 320, 330. The ultrafiltrate pump 350 pumps a regulated amount of spent dialysate (ultrafiltrate) through a check valve 391. The check valve 391 prevents fluid from entering the Ultrafiltrate Subsystem. After the check valve, the fluid enters the UF waste valve 392. This ultrafiltrate is the fluid taken off the patient through hemodialysis. The spent dialysate leaves the balance chambers 320, 330 through balance chamber valve 1 311 and balance chamber valve 5 315. After exiting the balance chamber valves, the spent dialysate flows through a check valve 390. The check valve 390 prevents fluid from the drain manifold entering the Ultrafiltrate Subsystem. The spent dialysate then enters the UF waste valve 392. The UF waste valve 392 channels the waste fluid either to the drain manifold 185 or to the ACDF Subsystem 397. By diverting the spent dialysate back to the ACDF, it is purified in the Active Carbon Filter and reused as fresh dialysate. Thus, the amount of fluid required to perform the treatment is reduced. The UF waste valve 392 is controlled and driven by the UF waste valve control circuits and software 393.

The ACDF system reduces the amount of fluid required for a hemodialysis system be filtering and reusing the spent dialysate. The spent dialysate is run back through the active carbon filter and the solutes are substantially removed to make fresh dialysate which is then used again for the dialysis treatment.

It is noted that substantially less fluid is needed to perform a dialysis procedure because the spent dialysate is recycled through the active carbon filter. Using an active carbon filter to purify spent dialysate, thus dramatically reducing the amount of water required to perform a hemodialysis treatment.

Universal Active Carbon Dialysate Filter Machine.

An example of a universal active carbon dialysate filter machine is disclosed, including a pre-treatment subsystem; an active carbon filter subsystem; a dialysate preparation subsystem; an ultrafiltrate subsystem; a dialyzer subsystem; a blood handling subsystem; a mechanical subsystem; and an electrical and software subsystem.

The Universal ACDF System is a water purification, dialysate preparation, and dialysate filtration system. It can generate enough dialysate for a full hemodialysis treatment from: a fixed volume of starter fluid (may be drinking water or a volume of bagged medical fluid), a concentrated dialysate solution, and spent dialysate solution. It can work with any dialysis machine that accepts a fresh dialysate input and has a spent dialysate output.

Water purification devices on the market today only purify water. There isn't a device that accepts spent dialysate. independent of the hemodialysis machine, and then purifies the water so that this water can be reused in a treatment. Sorbent technologies offer a similar outcome, but do so with a consumable component—the sorbent filter. All sorbent systems are integrated with a hemodialysis machine. The device described in this disclosure is independent of any hemodialysis machine. The device also uses a reusable filter rather than a consumable filter. The device also uses the active carbon filter technology, which is currently not used in any commercial dialysis device.

In an example, the pretreatment subsystem, dialysate preparation subsystem, mechanical subsystem, and electrical and software subsystem are as already described above for the integrated hemodialysis machine.

In an example, the Universal ACDF Machine does not include the dialyzer subsystem or blood handling subsystem.

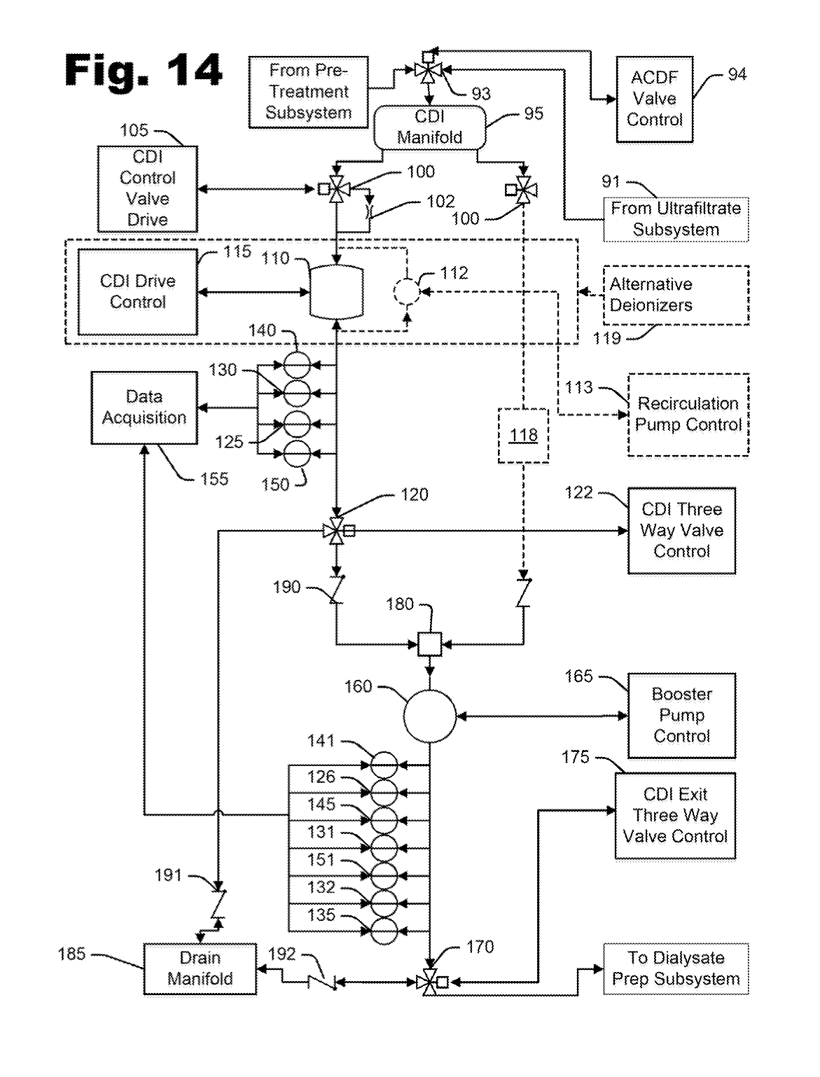

FIG. 14 shows an example active carbon dialysate filter subsystem of the Universal ACDF machine. In an example, an active carbon filter subsystem includes a Spent Dialysate from Ultrafiltrate Subsystem 91; an Active Carbon Dialysate Filter (ACDF) Control Valve 93; and an ACDF Valve Control Circuits and Software 94 (e.g., a three way valve). Spent dialysate 91 enters the ACDF Subsystem from the Ultrafiltrate Subsystem. The ACDF Control Valve 93 is a three way valve that switches between one of two inputs —pre-treated water 94 and spent dialysate 91. The ACDF valve 93 is controlled and driven by the ACDF Valve Control Circuits and Software 94. The selected fluid enters the CDI manifold 95 that divides the flow in to one or more fluid paths. One fluid path is demonstrated in the figure. but zero or more CDI fluid circuits 118 comprising the fluid circuit description below may be added.

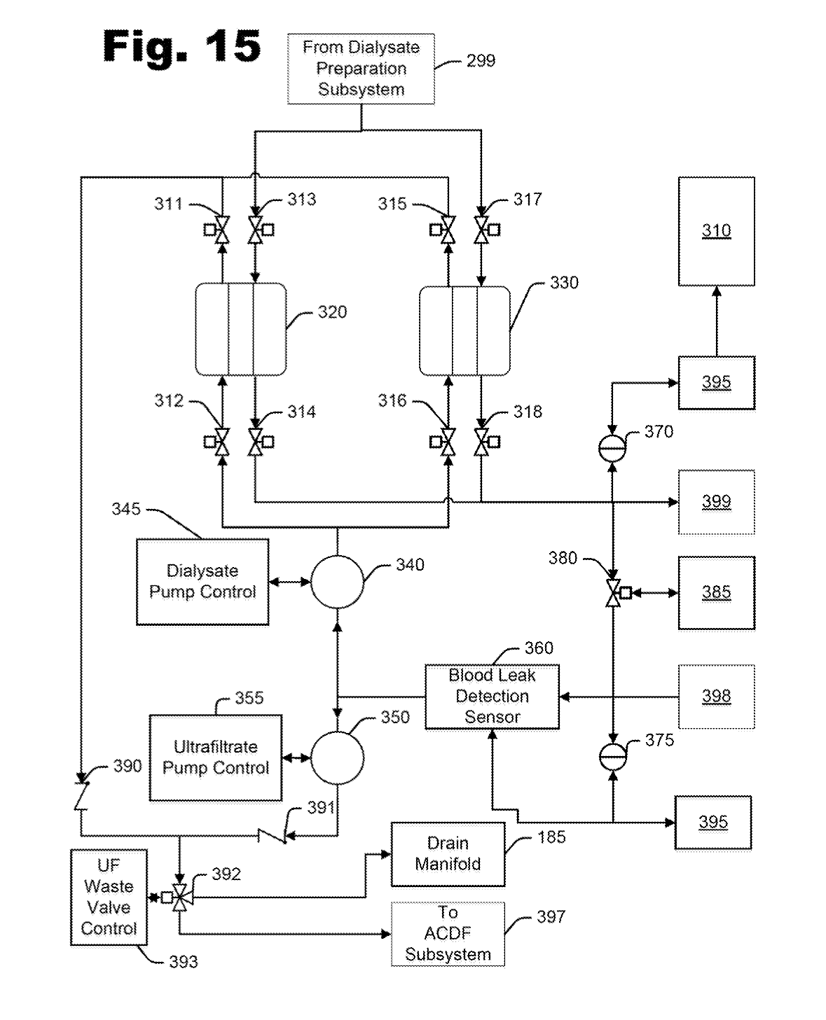

FIG. 15 shows an example ultrafiltrate subsystem of the Universal ACDF machine. The ultrafiltrate subsystem includes UF Waste Valve 392, UF Waste Valve Control Circuits and Software 393, DAQ Circuits and Software 395, Spent Dialysate to ACDF Subsystem 397, Spent Dialysate from Hemodialysis Machine 398, Fresh Dialysate to Hemodialysis Machine 399.

After exiting the balance chamber valves, the pressure of the fresh dialysate is measured by the fresh dialysate dialyzer pressure sensor 370. The sensor output is captured by the data acquisition circuits and software 395. The fresh dialysate then enters the Hemodialysis Machine 399. Spent dialysate is received from the Hemodialysis Machine 398.

The Hemodialysis Machine is any commercial dialysis machine that accepts an input of fresh dialysate and outputs spent dialysate. After the blood leak detection sensor 360, the spent dialysate flows in to two pumps, the dialysate pump 340 and the ultrafiltrate pump 350. The dialysate pump 340 pumps spent dialysate in to the balance chambers 320, 330. The ultrafiltrate pump 350 pumps a regulated amount of spent dialysate (ultrafiltrate) through a check valve 391. The check valve 391 prevents fluid from entering the Ultrafiltrate Subsystem. After the check valve, the fluid enters the UF waste valve 392. This ultrafiltrate is the fluid taken off the patient through hemodialysis.

The spent dialysate leaves the balance chambers 320, 330 through balance chamber valve 1 311 and balance chamber valve 5 315. After exiting the balance chamber valves, the spent dialysate flows through a check valve 390. The check valve 390 prevents fluid from the drain manifold entering the Ultrafiltrate Subsystem. The spent dialysate then enters the UF waste valve 392.

The UF waste valve 392 channels the waste fluid either to the drain manifold 185 or to the ACDF Subsystem 397. By diverting the spent dialysate back to the ACDF, it is purified in the Active Carbon Filter and reused as fresh dialysate. Thus, the amount of fluid required to perform the treatment is reduced. The UF waste valve 392 is controlled and driven by the UF waste valve control circuits and software 393.

The Universal ACDF system is a dialysate generator and recycler. From a fixed, small volume of starter fluid, the system may generate enough dialysate for a full hemodialysis treatment (up to 200 liters). It is compatible with any machine that accepts fresh dialysate input and outputs spent dialysate.

The Universal ACDF system reduces the amount of fluid required for a hemodialysis system be filtering and reusing the spent dialysate. The spent dialysate is run back through the active carbon filter and the solutes are substantially removed to make fresh dialysate which is then used again for the dialysis treatment.

The Universal ACDF system uses substantially less fluid to perform a dialysis procedure because the spent dialysate is recycled through the active carbon filter. The Universal ACDF system may also use an active carbon filter to purify spent dialysate, thus dramatically reducing the amount of water required to perform a hemodialysis treatment. The Universal ACDF system may also use an active carbon filter to purify water from drinking water to water for dialysis. The Universal ACDF system provides a universal water purification and dialysate purification device.

It is noted that the examples shown and described are provided for purposes of illustration and are not intended to be limiting. Still other examples are also contemplated.

1. A hemodialysis system, comprising:
a heat disinfected dialysate fluid path;
a disposable blood fluid path; and
an active carbon filter to purify influent water to purified water for dialysis or as a substitution fluid.

2. The hemodialysis system of claim 1, further comprising a plurality of subsystems configured such that spent dialysate is filtered by active carbon filter and reused as dialysate for hemodialysis.

3. The hemodialysis system of claim 1, further comprising a plurality of subsystems configured as a single machine to purify the influent water by active carbon filter and hemodialysis treatment, wherein the system provides heat disinfection of a dialysis machine with active carbon filter by the same component used for water purification.

5. A water purification method for hemodialysis, comprising:
providing a heat disinfected dialysate fluid path;
providing a disposable blood fluid path; and
purifying influent water via an active carbon filter for dialysis or as a substitution fluid.

6. The water purification method of claim 5, further comprising water purification modules sized with relation to the contaminant levels in the feed water.

7. The water purification method of claim 5, further comprising providing bagged water or dialysate solutions.

8. The water purification method of claim 5, further comprising determining the fluid flow rate from at least one bag.

9. The water purification method of claim 5, further comprising heating purified water before passing it to a dialysate module.

10. The water purification method of claim 5, further comprising receiving already purified water and passing the already purified water to the dialysate module.

11. The water purification method of claim 10, wherein the already purified water is heated before passing to the dialysate module.

12. The water purification method of claim 5, further comprising mixing concentrated dialysate with purified water from the water module, providing volumetric control of dialysate to and from the dialyzer, and monitoring ultrafiltrate removal from a patient.

13. A modular dialysis system, comprising:
a dialysate module;
a common therapy module; and
a fluid source module to provide water for hemodialysis, dialysate, dialysis fluid, or on-line substitution quality fluid;
wherein each of the modules are integrated with a dialysis machine, and wherein the modules enable changing a purpose of the dialysis machine without changing user interfaces.

14. The modular dialysis system of claim 13, further comprising a single common user interface for multiple types of dialysis.

15. The modular dialysis system of claim 13, wherein a malfunctioning module is swappable for a new module without having to repair the entire dialysis machine.

16. The modular dialysis system of claim 13, wherein the dialysis machine is reconfigurable by replacing one of the modules without reconfiguring the entire dialysis machine.

17. The modular dialysis system of claim 13, wherein the fluid source module is configured to provide bagged fluids or dialysate solutions.

18. The modular dialysis system of claim 17, wherein the fluid source module determines the fluid flow rate from at least one bag.

19. The modular dialysis system of claim 13, wherein the fluid source module heats fluid before passing it to the dialysate module.

20. The modular dialysis system of claim 13, wherein the fluid source module receives already purified water and passes it to the dialysate module.

21. A dialysis system, comprising:
a heat disinfected dialysate fluid path;
an active carbon filter to purify influent water to purified water for dialysis or as a substitution fluid.

22. The dialysis system of claim 21, further comprising a plurality of subsystems configured such that spent dialysate is filtered by active carbon filter, combined with concentrated dialysate components, and reused as dialysate for hemodialysis.

23. The dialysis system of claim 21, further comprising a plurality of subsystems configured as a single machine to purify the influent water by active carbon filter and dialysis treatment, wherein the system provides heat disinfection of a dialysis machine with active carbon filter by the same component used for water purification.
